(19) United States
(12) Patent Application Publication
(10) Pub. No.: US 2024/0268875 A1
(43) Pub. Date: Aug. 15, 2024

(54) METHODS AND DEVICES FOR ORTHOPEDIC SURGICAL TECHNIQUES

(71) Applicant: SUMMA HEALTH SYSTEM, Akron, OH (US)

(72) Inventors: Eric MILLER, Medina, OH (US); Trinity Kronk SAMSON, Lakewood, OH (US); Muhammad Saiyed ISLAM, Lilburn, GA (US); Alyssa Anastacia BASDAVANOS, Mineral Ridge, OH (US)

(73) Assignee: SUMMA HEALTH SYSTEM, Akron, OH (US)

(21) Appl. No.: 18/567,761

(22) PCT Filed: Aug. 11, 2022
(86) PCT No.: PCT/US2022/040090

§ 371 Date: Dec. 6, 2023

Related U.S. Application Data
(60) Provisional application No. 63/231,981, filed on Aug. 11, 2021.

(51) Int. Cl. A61B 17/88; A61B 17/82

(52) U.S. Cl. CPC A61B 17/8861; A61B 17/82; A61B 17/8863; A61B 17/8869

(57) ABSTRACT

A device for passing cerclage wire includes a first member having a first region and a second region, where the first member includes a cannula formed therein. The first region is substantially linear in shape and the second region is hooked. The first region includes at least one aperture that is fluidly connected to the cannula, wherein the at least one aperture is configured to receive cerclage wire. Kits including the device and methods employing the device are also disclosed.

### RELATED APPLICATIONS

This application claims priority to and the benefit of co-pending U.S. Provisional Patent Application 63/231,981, Attorney Docket No. 116449-0400, entitled “METHODS AND DEVICES FOR MANIPULATING CERCLAGE WIRE” with the filing date of Aug. 11, 2021, which is herein incorporated by reference in its entirety.

### BACKGROUND

The present disclosure relates generally to the field of orthopedic surgical techniques, and more specifically to devices and methods for orthopedic cerclage wire passing and binding.

Generally, cerclage wire is used in orthopedic surgical operations to facilitate fixation or stabilization of various anatomical features. Often, passing cerclage wire through a surgical site can cause damage to tissue resulting from the required size of the incision often necessitated by the need for visibility of the wire to capture it on the opposite side of the bone or anatomical feature. This can lead to various medical complications and affect overall healing and recovery of the surgical site. In other instances, devices used to pass cerclage wire can be cumbersome and difficult to operate, which extends surgery duration and increases likelihood of unintentional damage to soft tissue.

Accordingly, it would be advantageous to provide a device for passing cerclage wire that is easy to use and is minimally invasive.

### SUMMARY

One aspect of the present disclosure relates to a device for passing cerclage wire including a first member having a first substantially linear region and a second hooked region, the first member having a cannula formed therein. The device includes a second member having a third substantially linear region and a fourth hooked region, the second member having a second cannula formed therein. The first region includes at least one aperture that is fluidly connected to the cannula and configured to receive cerclage wire, and the third region includes at least one aperture fluidly connected with the second cannula.

In embodiments, terminal ends of the second region and the fourth region are configured to be received together.

In embodiments the device includes a telescoping member coaxially disposed in an overlapping arrangement with one of the second region or the fourth region when the device is in an open configuration. The telescoping member is slidably displaceable from the one of the second region or the fourth region to at least partially overlap with the other when the device is in a closed configuration.

In embodiments the device includes handles connected to the first member and second member. When a user interacts with the handles the device is alternately placed in an open configuration or a closed configuration.

In embodiments, the device includes a locking mechanism disposed between the first region and the third region. The locking mechanism include a grooved member coupled to the first region, and a protruding member coupled to the third region. The protruding member is configured to be received within the grooved member, where the protruding member is slidably engaged with the grooved member.

In embodiments, the device includes a joint pivotably coupling the first member and the second member. Specifically, the first member connects to the pivot between the first region and the second region, and the second member connects to the pivot between the third region and the fourth regions. The device may further include at least one handle rotatably coupled to the joint. Specifically, rotating the at least one handle causes the first member and the second member to pivot about the joint such that a distance between a distal end of the first member and a distal end of the second member changes.

In another aspect of the disclosure, a surgical kit for stabilizing one or more anatomical features within a surgical site includes: a first device for passing cerclage wire; a second device for tensioning cerclage wire; a third device for cutting cerclage wire; and optionally a fourth device for securing cerclage wire. The first device includes a first cannulated member having a first region and a second region, and a second cannulated member having a third region and a fourth region. Specifically, each of the first and third regions are substantially linear in shape and each of the second and fourth regions are hooked, each of the first and third regions including at least one aperture disposed therein and configured to receive cerclage wire. The second device includes a body having an inner channel disposed therein, and a locking mechanism disposed within the channel configured to slidably displace within the channel along an axis of the body, where displacement of the locking mechanism tensions a cerclage wire. The third device includes a first shaft; a second shaft coaxially disposed within the first shaft; and a handle coupled to the first shaft. The distal end of the first shaft includes a cutting interface configured to cut cerclage wire passing through the first shaft and the second shaft.

In another aspect of the disclosure, a method for stabilizing anatomy within a surgical site includes providing a first device having elongated first and second members, each having a substantially linear region with a cannula formed within. The method includes passing a cerclage wire through the surgical site using the first device and providing a second device having a locking mechanism disposed within a channel. The method includes passing a terminal end of the cerclage wire into the locking mechanism of the second device and tensioning the cerclage wire by displacing the locking mechanism. The method includes securing a first portion adjacent the terminal end of the cerclage wire using the third device, and cutting the terminal end of the cerclage wire using a fourth device.

### BRIEF DESCRIPTION OF THE DRAWINGS

The disclosure will become more fully understood from the following detailed description, taken in conjunction with the accompanying figures, wherein like reference numerals refer to like elements, in which:

FIG. 1 is a perspective, rear side view of an exemplary cable passing device in a closed configuration.

FIG. 2 is a perspective, rear side view of the cable passing device of FIG. 1 in an open configuration.

FIG. 3 is a schematic representation of a side view of a cable passing device having a telescoping cannula, according to an exemplary embodiment.

FIG. 4 is a schematic representation of a side view of a cable passing device having a telescoping cannula, according to another exemplary embodiment.

FIG. 5 is a perspective view of another embodiment of a cable passing device of in an open configuration.

FIG. 6 is a perspective view of the cable passing device of FIG. 5 in a closed configuration.

FIG. 7 is a schematic representation of an end view of the sliding mechanism within the cable passing device of FIG. 5, according to an exemplary embodiment.

FIG. 8 is a schematic representation of a side view of a cable passing device having expandable curved cannulas shown in an open configuration, according to an exemplary embodiment.

FIG. 9 is a schematic representation of a side view of the cable passing device of FIG. 8 in a closed configuration.

FIG. 10 is a schematic representation of the cable passing device of FIG. 9, shown without a handle.

FIG. 11 is a side perspective view of a cable passing device having a hinge mechanism, according to an exemplary embodiment.

FIG. 12 is a side perspective view of a cable passing device having a pivot hinge mechanism shown in disconnected configuration, according to an exemplary embodiment.

FIG. 13 is a side perspective view of the cable passing device of FIG. 12 in a connected configuration.

FIG. 14 is lengthwise cross-sectional view of a cable tensioning device, according to an exemplary embodiment.

FIG. 15 is a schematic representation of a sectional view of the cable tensioning device of FIG. 14 including a ratchet handle, according to an exemplary embodiment.

FIG. 16 is a side view of a cable securement device, according to an exemplary embodiment.

FIGS. 17-19 are alternate perspective views of a cable cutting device, according to an exemplary embodiment.

FIG. 20 is a lengthwise cross-sectional view of the cable cutting device of FIG. 18.

FIG. 21 is a schematic representation of a cerclage assembly, according to an exemplary embodiment.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

Referring generally to the figures, exemplary suitable cerclage passing devices are shown. As described above, internal fixation devices such as cerclage wire (“cable”) are used in various surgical applications to stabilize one or more anatomical features. During use, these devices are inserted into a surgical site and navigated around one or more anatomical features. In the case of a wire, it is bound (e.g., cut, crimped, or otherwise terminated) to secure the wire and the anatomical features it surrounds in place. In various implementations, cerclage wire can be used to stabilize bone fractures (e.g., of long bones), to secure terminal ends of soft tissue or organs, or to secure other soft tissue or hard tissue.

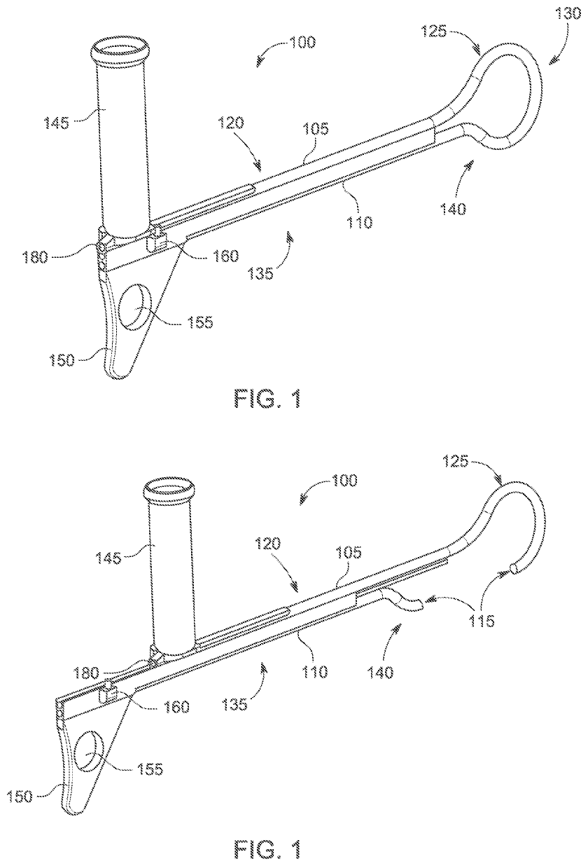

Referring to FIGS. 1 and 2, an exemplary passing device 100 is shown in a closed (FIG. 1) and open (FIG. 2) configuration. The device 100 is configured to be inserted within a surgical site to facilitate passing, for example, of cerclage wire (“cable”) through the surgical site to enable fixation or stabilization of one or more anatomical features therein. The device 100 includes a first member 105 axially movable relative to a second member 110, each of which having a cannula 115 within. The cannula 115 has an inner diameter that is adapted to enable passage of a cable through the members 105, 110. In various embodiments, the inner diameter of the cannula 115 may be between approximately 5 mm to approximately 8 mm. The first member 105 is structured to have a first region 120 and a second region 125, where the second region 125 includes at least a partial hooked or looped portion 130. The first region 120 may be substantially linear or may be ergonomically contoured to adapt to one or more users of the device 100. The second member 110 is structured to have a first, substantially linear, region 135 arranged parallel to the first region 120 of the first member 105. At a terminal, distal, region 140, the second member 110 angles away to meet portion 130 and align cannula 115 providing a substantially continuous pathway between the first member 105 and the second member 110. In practice, portion 130 may be connected to the second member 110 rather than the first member 105 (as shown).

The first member 105 further includes a handle grip 145, shown as being cylindrical although other handles would be usable. The second member 110 includes a second handle 150, shown as an extension with an opening 155 to accommodate a user's finger, for example, although handle 145 and second handle 150 are merely two representations of a variety of possible elements whose function is to provide a sliding mechanism and/or a sliding lock mechanism for relative movement of the members 105, 110 operable by a user or users.

In various embodiments, the cable may be inserted into the cannula 115 via one or more apertures (e.g., holes, slots, etc.) disposed within the first region 120 of the first member 105. In some embodiments, the cable may be inserted at or near a terminal end 180 of the first region 120 of the first member 105. In yet other embodiments, the first member 105 may include multiple insertion points to facilitate inserting the cable into the cannula 115.

In practice, a user would release a spring catch 160 and manipulate the handle 145 and extension 150 causing the first member 105 and second member 110 move axially relative to each other alternatingly joining ends of the first member 105 and second member 110 establishing continuity in the cannula 115 in the closed configuration (FIG. 1) and spacing ends of the first member 105 and second member 110 and establishing a discontinuity in the cannula 115 in the open configuration (FIG. 2). When the device 100 is in the closed configuration (FIG. 2), the cable within the cannula 115 may circumnavigate one or more anatomical features within the surgical site.

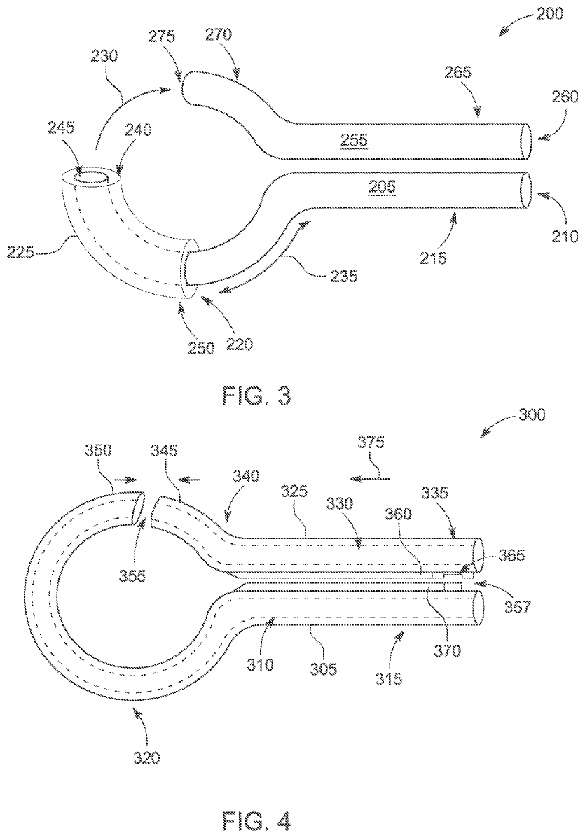

Referring to FIG. 3, a schematic representation of a telescopic cable passing device 200 is shown, according to an exemplary embodiment. The device 200 is configured to be inserted within a surgical site to facilitate passing cerclage wire (“cable”) through the surgical site to enable fixation or stabilization of one or more anatomical features therein. The device 200 includes a member 205, which has a cannula 210. The cannula 210 has an in inner diameter that is adapted to enable passage of a cable through the member 205. In various embodiments, the inner diameter of the cannula 210 may be between approximately 5 mm to approximately 8 mm. The member 205 is structured to have a first region 215 and a second region 220, where the second, hooked region 220 includes a telescoping portion 225. The first region 215 may be substantially linear or may be ergonomically contoured to adapt to one or more users of the device 200. The telescoping portion 225 is arranged coaxially with the hooked region 220 and is configured to slide relative to the hooked region 220 along a telescopic pathway 230. In various embodiments, the telescoping portion 225 may be arranged within an inner region or an outer region of the hooked region 220 (i.e. inner or outer concentric). Accordingly, when the telescoping portion 225 is in a retracted configuration, an end 240 of the telescoping portion 225 disposed furthest from the first region 215 is substantially even with an end 245 of the hooked region 220. When the telescoping portion 225 is in an expanded configuration, an end 250 of the telescoping portion 225 disposed nearest the first region 215 is disposed adjacent the end 245 of the hooked region 220 to form an arched structure configured to facilitate navigating cable about one or more anatomical features (e.g., a femur). In various embodiments, the telescoping portion 225 is structured to extend by 90 degrees or more such that the device 200 may circumnavigate within the range of 180 to 270 degrees. In various embodiments, the telescoping portion 225 is configured to extend by less than 90 degrees. In yet other embodiments, the telescoping portion 225 is configured to circumnavigate up to approximately 360 degrees.

In various embodiments, extension and/or retraction of the telescoping portion 225 may be facilitated by an actuator 235 disposed adjacent to at least one of the hooked region 220 or the first region 215. In various embodiments, the actuator 235 may be a linear slide coupled to the member 205. For example, the actuator 235 may be a thumb-driven member (e.g., flexible rod) that slidably engages with the member 205 (e.g., within a groove or track) such that a force applied to an end of the thumb-driven member causes the actuator 235 to slide relative to the member 205 and force the telescoping portion 225 to extend through the telescoping pathway 230. Accordingly, when a linear force is supplied to the actuator 235 (e.g., by a user's thumb or finger), the telescoping portion 225 is extended. In various embodiments, the end 240 of the telescoping portion 225 furthest from the first region 215 may be blunted to facilitate ease of insertion within a surgical site. In various embodiments, the end 240 may include a removable cap. In yet other embodiments, the telescoping portion 225 may include multiple telescoping members, each disposed coaxially and structured to expand and retract along the telescopic pathway 230 to facilitate circumnavigation of one or more anatomical features.

During use, cable may be passed through the cannula 210 of the member 205 and the ends 240, 245 may be inserted into a surgical site (e.g., within an incision or other opening).

In various embodiments, the cable may be inserted into the cannula 210 via one or more apertures (e.g., holes, slots, etc.) disposed within the first region 215 of the member 205. In some embodiments, the cable may be inserted at or near a terminal end of the first region 215 of the member 105. In yet other embodiments, the member 205 may include multiple insertion points to facilitate inserting the cable into the cannula 210. The actuator 235 may then be used to extend the telescoping portion 225 along the telescopic pathway 230, which enables the cable within the cannula 210 to circumnavigate one or more anatomical features within the surgical site. In optional embodiments, the device 200 may include only a single member 205, thus requiring only small openings resulting in a minimally invasive device.

In some instances, a cable passing device may include multiple members. Continued reference to FIG. 3 also shows the device 200 including a second member 255, which is configured to be structurally complementary to the member 205, where the second member 255 is adapted to be used cooperatively with the member 205 to circumnavigate a cable about one or more anatomical features (e.g., a femur). As shown, the member 255 also includes a cannula 260, which is structured to accommodate cerclage wire (“cable”) therein. The member 255 includes a first region 265 and a hooked region 270, which terminates at an end 275 distal to the region 265. In various embodiments, the end 275 may be tapered or blunted. In various embodiments, the region 265 may be substantially linear. In other embodiments, the region 265 may be ergonomically formed to facilitate ease of use by one or more users of the device 200.

During use, the first member 205 of the device 200 may be inserted within a surgical site (after a cable is passed through the cannula 210). The actuator 235 may then displace the telescoping portion 225 through the telescopic pathway 230 such that the end 240 of the telescoping portion 225 is disposed substantially adjacent to the end 245 of the hooked region 220. Once the telescoping portion 225 is extended, the second member 255 may be inserted within the surgical site. Accordingly, the end 240 of the telescoping portion 225 may be joined with the end 275 of the second member 255, which may enable cable from within the cannula 210 to be passed through the cannula 260 of the second member 255. In various embodiments, the second member 255 may include one or more apertures disposed within the region 265 to allow the cable to enter into or exit from the cannula 260. In various embodiments, at least one of the first member 205 or the second member 255 may be ergonomically formed to facilitate ease of handling and use.

In various embodiments, the device 200 may include one or more locking mechanisms to facilitate alignment and engagement between the members 205, 255. For example, the device 200 may include a locking mechanism formed between the first region 215 of the member 205 and the first region 265 of the member 255 such that when the member 255 is inserted within the surgical site, the member 255 may be engaged with the member 205. In various embodiments, the end 275 of the member 255 may have a smaller diameter compared to the end 240 of the telescoping portion 225 such that when the telescoping portion 225 is extended, the end 240 may concentrically fit over the end 275 to facilitate ease of cable passage. In various embodiments, the telescoping portion 225 is configured to extend through at least 90 degrees of circumnavigation. In other embodiments, the telescoping portion 225 may extend at least 180 degrees or at least 270 degrees. In various embodiments, the hooked region 220 and the telescoping portion 225 form a first arc and the hooked region 270 forms a second arc such that when the end 240 is adjacent or coupled to the end 275, the first arc and the second arc substantially surround (e.g., circumferentially) an anatomical feature (e.g., a long bone) therein. In various embodiments, the first arc and the second arc at least partially surround the anatomical feature.

In some embodiments, a cable passing device may have stationary or “solid state” components to facilitate circumnavigation of cerclage wire (“cable”) through a surgical site. FIG. 4 shows a schematic representation of a side view of a cable passing device 300, according to an exemplary embodiment. The device 300 includes a first member 305 having a cannula 310. The member 305 includes a first region 315 and a second, hooked region 320. In various embodiments, the region 315 may be substantially linear. In other embodiments, the region 315 may be ergonomically formed to facilitate ease of use by one or more users of the device 300. In various embodiments, the first region 315 of the member 305 includes one or more apertures (e.g., holes, slots, etc.) disposed through an outer wall surrounding the cannula 310 to facilitate entry and/or exit of cable into the cannula 310. Although FIG. 4 shows the hooked region 320 extending or arching through approximately 270 degrees of rotation, the hooked region 320 may extend through any amount of rotation. The device 300 further includes a second member 325, which is configured to be complementary to the member 305. The second member 325 has a cannula 330 and includes a first region 335 and a second, hooked region 340. The first region 335 may be substantially linear or may be ergonomically formed to facilitate ease of use and handling. In various embodiments, the region 335 of the member 325 includes one or more apertures (e.g., holes, slots, etc.) disposed through an outer wall surrounding the cannula 310 to facilitate entry and/or exit of cable from the cannula 310.

The first member 305 is configured such that the hooked region 320 terminates at an end 350 and the second member 325 is configured such that the hooked region 340 terminates at an end 345. In various embodiments, at least one of the ends 345, 350 may be tapered or blunted. The members 305, 325 are structured such that the cannulas 310 and 330 are aligned when the ends 345 and 350 are disposed adjacent to each other (i.e., when a distance 355 between the ends 345, 350 is minimized). To facilitate alignment of the members 305, 325, the device 300 may include a sliding lock mechanism 357 disposed between the regions 315, 335. As shown in FIG. 4, the second member 325 includes a grooved member 360, which includes a central groove or channel 365, which is configured to receive a protruding member 370 included within the first member 305. Accordingly, the second member 325 may be aligned relative to the first member 305 by sliding the protruding member 370 within the groove 365. In various embodiments, at least one of the grooved member 360 or the protruding member 370 may include one or more locking features, which may be configured to prevent sliding between the members 305, 325.

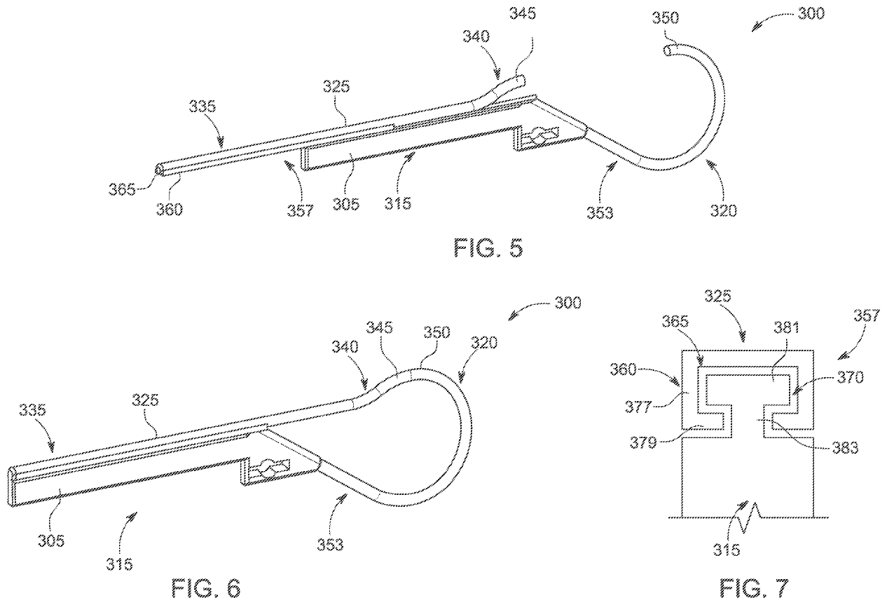

FIGS. 5 and 6 show perspective views of the device 300 in open and closed configurations, respectively. As shown, the member 325 is slidably engaged with the member 305 via the grooved members 360 and the protruding member 370 within the mechanism. In various embodiments, the member 325 may slide relative to the member 305 to move the end 345 near to the end 350 to change a configuration of the device 300 from an open configuration, as shown in FIG. 5, to a closed configuration, as shown in FIG. 6.

During use of the device 300, cable may be inserted within the cannula 310 of the first member 305. In various embodiments, the cable may be insertable or removable at one or more locations (e.g., within one or more holes, slots, or other apertures) along the first region 315. The first member 305 may be inserted into a surgical site and maneuvered such that the end 350 circumnavigates one or more anatomical features (e.g., femur). In various embodiments, the first and second members 305, 325 may be coupled via the mechanism 357 prior to or subsequent to insertion of the member 305, where the protruding member 370 is received within the groove 365 and retained therein via the grooved member 360. In some embodiments, after coupling of the members 305, 325, the member 305 may be inserted into the surgical site while the device 300 is in the open configuration (e.g., as shown in FIG. 5). In other embodiments, the member 305 may be inserted into the surgical site and the member 325 may be coupled to the member 305 via the mechanism 357. Once the first member 305 has been suitably positioned within the surgical site, the second member 325 may be engaged with the first member 305 via the members 360, 370. The second member 325 may then be slidably displaced relative to the first member 305 until the end 345 is disposed adjacent the end 350 and the device 300 is in the closed configuration (e.g., as shown in FIG. 6), which enables cable passage from the first member 305 through the second member 325. In various embodiments, the end 345 may have a smaller diameter than the end 350 such that when the members 305, 325 are aligned, the end 350 fits over the end 345. In other embodiments, the end 350 may have a smaller diameter than the end 345 and the end 345 may fit over the end 350. In various embodiments, the hooked region 320 and the forms a first arc and the hooked region 340 forms a second arc such that when the end 350 is adjacent or coupled to the end 345, the first arc and the second arc substantially surround (e.g., circumferentially) an anatomical feature (e.g., a long bone) therein. In various embodiments, the first arc and the second arc at least partially surround the anatomical feature.

In various embodiments, at least one of the members 305 or 325 may include one or more contoured portions, which are adapted to facilitate ease of insertion of the device 300 into the surgical site. For example, as shown in FIGS. 5 and 6, the member 305 may include a sloped region 353 disposed between the first region 315 and the hooked region 320, which may be configured to facilitate ease of insertion of member 305 into the surgical site. FIG. 7 shows an end view of the mechanism 357 of the device 300. In various embodiments, the grooved member 360 includes two mirroring elongated portions 377, which are configured to slide relative to the protruding member 370. As shown, the protruding member 370 may include a first portion 381 and a second portion 383, where the first portion is configured to be received within the groove 365. As shown, the first portion 381 may have a greater width compared to the second portion 383. Accordingly, a lip portion 379 of the grooved member 360 may engage with the second portion 383 to retain the protruding member 370 within the groove 365. In various embodiments, the protruding member 370 may be configured as a separate component coupled to the member 305. In other embodiments, the protruding member 370 may be integrally formed within the first region 315 of the member 305. Similarly, in various embodiments, the grooved member 360 may be configured as a separate component and coupled to the member 325. In other embodiments, the grooved member 360 may be integrally formed within the member 325. In yet other embodiments, the mechanism 357 (or an equivalent thereof) may be incorporated within other embodiments such as devices 100, 200, etc.

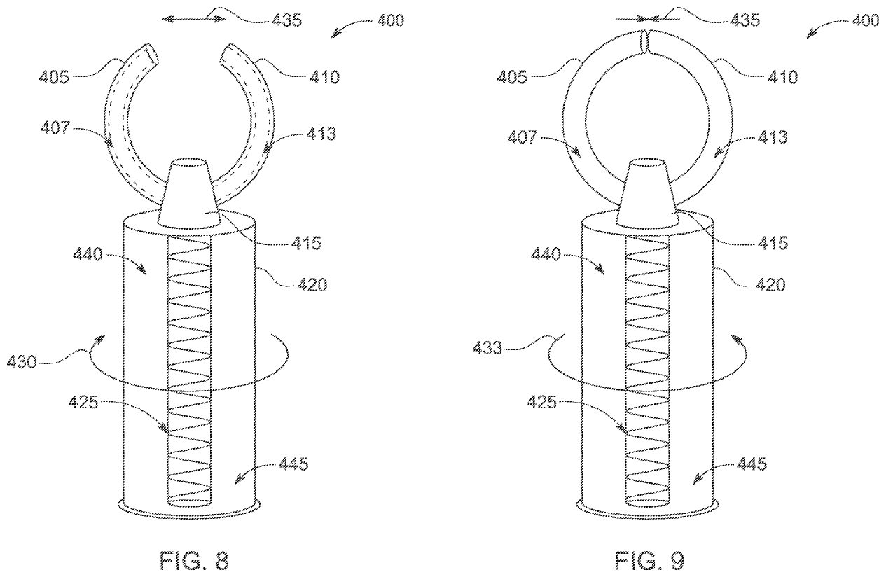

In some embodiments, a cable passing device may include retractable cannulas, which may articulate in a claw-like fashion to circumnavigate one or more anatomical features. FIGS. 8 and 9 show schematic representations of side views of a cable passing device 400, according to an exemplary embodiment. As shown, the device 400 includes a first curved member 405 having a first cannula 407 and a second curved member 410 having a second cannula 413. The first and second curved members 405, 410 are pivotably coupled at a first end via a shared hinge joint 415. Respective second ends of the first and second curved members 405, 410 are free to displace relative to each other such that when the device 400 is in an open configuration, a distance 435 between the second ends of the members 405, 410 is maximized and when the device 400 is in a closed configuration, the distance 435 between the second ends of the members 405, 410 is minimized or approximately zero. In various embodiments, the second ends of the members 405, 410 may be configured to engage in a concentrically overlapping arrangement, where one of the ends has a smaller diameter than the other end.

The device 400 includes a handle 420 which is rotatably coupled to the hinge 415. The handle 420 may include a threaded mechanism 425 (illustrated in cut-away to show mechanism) disposed therein such that rotation of the handle 420 in a first direction 430 (e.g., clockwise) may cause the device 400 toward the open configuration (as shown in FIG. 8) and rotation of the handle 420 in a second direction 433 (e.g., counterclockwise) opposite the first direction 430 may cause the device 400 toward the closed configuration (as shown in FIG. 9). As shown in FIGS. 8 and 9, the handle 420 may also include one or more channels disposed therein. For example, the handle 420 may include two channels where a first channel 440 is arranged on a first side of the threaded mechanism 425 and a second channel 445 is arranged on a second, opposing side of the threaded mechanism 425.

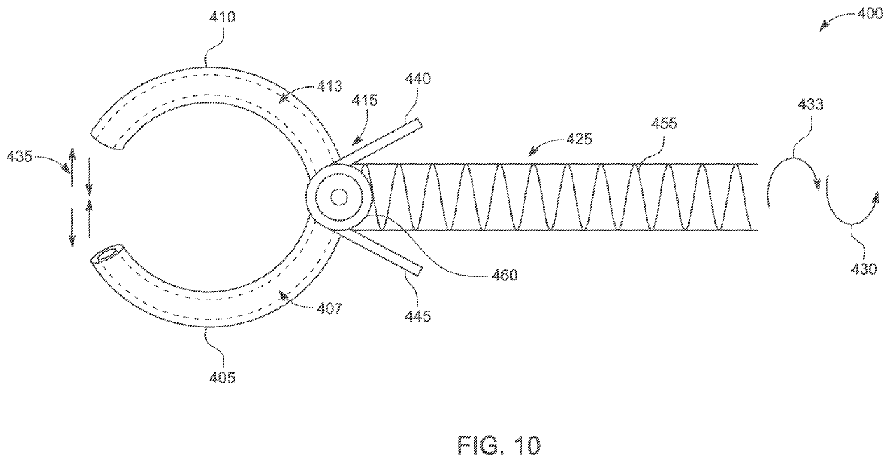

In various embodiments, the hinge joint 415 may be configured as a worm drive. As shown in FIG. 10, the hinge joint 415 may include a first member 460, which is configured to pivot the members 405, 410 about the hinge joint 415 when the first member 460 is rotated. In various embodiments, the first member 460 may be a worm wheel. The threaded mechanism 425 may include a threaded shaft (e.g., worm) 455, which is configured to engage with the first member 460. Accordingly, when the threaded shaft 455 within the threaded mechanism 425 is rotated in the direction 430 or the direction 433, the first member 460 rotates and engages with the members 405, 410 to adjust the distance 435 between the terminal ends of the members 405, 410. In various embodiments, at least one of the terminal ends of the members 405, 410 is blunted.

During use of the device 400, cerclage wire (“cable”) may be passed through one of the channels 440, 445 and into the first member 405. The device 400 may then be inserted within a surgical site and arranged such that the first member 405 is disposed on a first side of an anatomical feature and the second member 410 is disposed on a second side of the anatomical feature. For example, the device 400 may be inserted within a surgical site such that the first member 405 is disposed on an underside of a femoral bone and the second member is disposed on a top side of the femoral bone. Once the device 400 is positioned, the handle 420 may be rotated in the direction 433 to cause the members 405, 410 to pivot about the hinge joint 415 and minimize the distance 435. Once the device 400 is in the closed position, the cable may be passed through the second member 410 and through the other of the two channels 440, 445. The device 400 may then be disengaged by rotating the handle 420 in the direction 430 to cause the members 405, 410 to pivot about the hinge joint 415 and maximize the distance 435. Once in the open configuration, the device 400 may then be extracted from the surgical site.

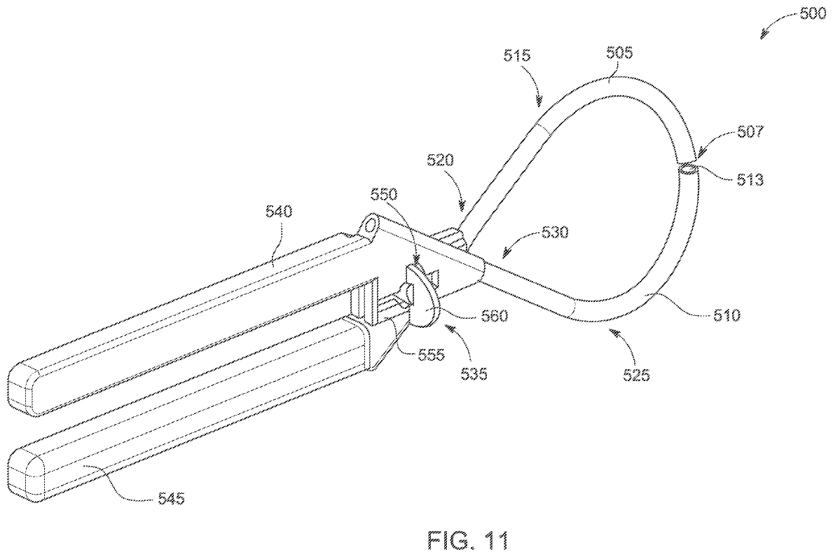

In some embodiments, a cable passing device may be configured to have a scissoring mechanism to facilitate circumnavigation within a surgical site. FIGS. 11-13 show perspective views of a cable passing device 500, according to an exemplary embodiment. As shown, the device 500 includes a first member 505 and a mirrored second member 510, where each of the members 505, 510 include respective cannulas disposed therein. The first member 505 includes a hooked region 515, which is defined between a distal end 507 and a proximal end 520. Similarly, the second member 510 includes a hooked region 525, which is defined between a distal end 513 and a proximal end 530. The proximal ends 520, 530 of the respective members 505, 510 are rotatably coupled at a pin or hinged joint 535. The proximal end 520 of the first member 505 is integrally formed with a first handle 540 and the proximal end 530 of the second member 510 is integrally formed with a second handle 545, where the handles 540, 545 are configured to facilitate articulation of the members 505, 510 about the joint 535. In various embodiments, at least one of the ends 507, 513 may be tapered or blunted.

As shown, the members 505, 510 may be disconnectable at the joint 535 via disengaging a lock mechanism 550. As shown, the lock mechanism 550 includes a rotatable key 560, which may be rotated relative to a slot 555 such that when the key 560 is in a first orientation, the lock mechanism 550 is engaged and when the key 560 is in a second orientation, the lock mechanism 550 is disengaged and the members 505, 510 may be separated. Accordingly, during use of the device 500, the ends 507 and 513 of the respective members 505, 510 may be inserted into a surgical site. The handles 540, 545 may be rotated about the joint 535 to cause opening and subsequent closure of the members 505, 510 about one or more anatomical features. A cable may then be passed through the cannula of the member 505 and subsequently through the cannula of the member 510. Once the cable is passed through the device 500, the handles 540, 545 may be again used to cause separation between the members 505, 510 and enable removal of the device 500 from the surgical site.

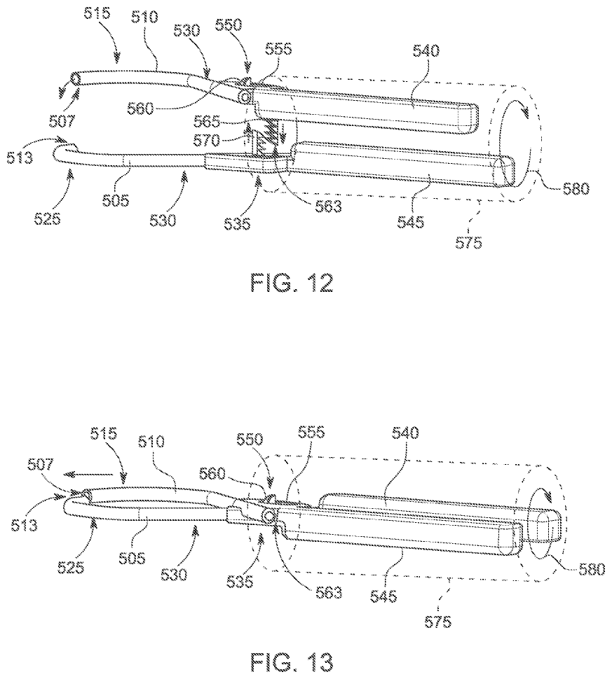

FIG. 12 shows a perspective view of the device 500 in a disconnected configuration. As shown, the device 500 may include a pivot or hinge joint 563, in addition to or instead of the joint 535. The joint 563 may be formed by engagement between a first engagement member 565 and a second, complementary engagement member 570. In various embodiments, the engagement members 565, 570 may be threadably, slidably, rotatably, magnetically, or otherwise mutually couplable. The members 565, 570 may be configured such that when aligned, the members 565, 570 may engage when subject to a torsional force in a direction perpendicular to a direction in which both members 565, 570 extend. In addition, the device 500 may include an outer sleeve or handle 575, which is configured to enclose and enable manipulation of the handles 540, 545. Accordingly, to connect the members 505, 510, the members 565, 570 may be aligned and the sleeve 575 may be rotated in a direction 580 to engage the members 565, 570. In some embodiments, the device 500 may not include the sleeve 575 and rotation of the members 565, 570 is facilitated by the handles 540, 545. Once the members 565, 570 are engaged, the members 505, 510 are rotatably connected about the joint 563, as shown in FIG. 13, such that the handles 540, 545 may be used to articulate the members 505, 510 relative to each other in a scissor-like motion.

During use of the device 500, the members 505, 510 may be inserted into a surgical site and arranged about one or more anatomical features disposed therein. The handles 540, 545 may be used to align the members 565, 570 and the sleeve 575 may be used to rotate the handles 540, 545 relative to each other, which may then cause engagement of the members 565, 570 to connect the members 505, 510. Once connected, the members 505, 510 may be articulated relative to each other about the joint 563 such that the ends 507, 513 are adjacent to facilitate passage of cable therebetween. In various embodiments, the end 507 may be configured to be received within the end 513. In other embodiments, the end 513 may be receivable within the end 507. Once cable is passed through the device 500, the sleeve 575 may again be used to rotate the handles 540, 545 about the joint 563 to disengage the members 565, 570, which allows separation of the members 505, 510. Once separated, the members 505, 510 may then be removed from the surgical site.

In various implementations, one or more cable tensioning devices may be used in conjunction with a cable passing device (e.g., devices 100, 200, 300, 400, 500) to facilitate tensioning cable that has been passed through the cable passing device, which aids in fixation or stabilization of one or more anatomical features within a surgical site.

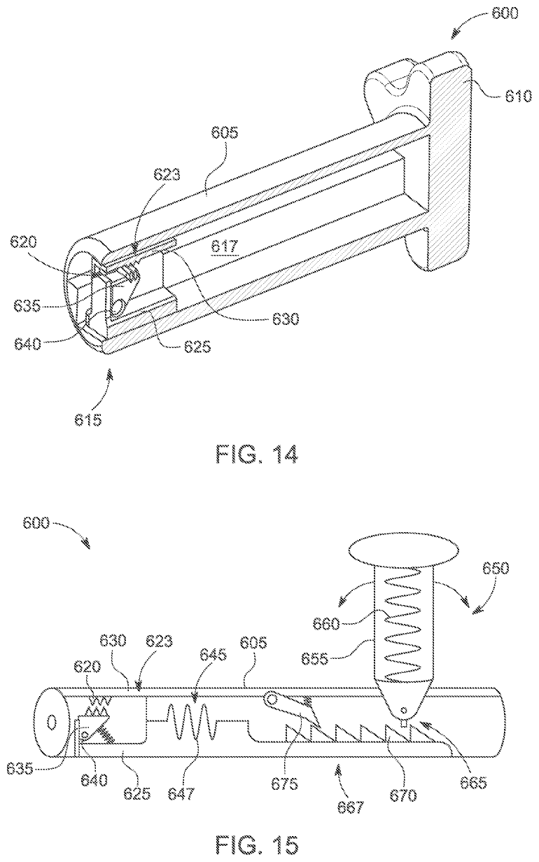

FIG. 14 shows a lengthwise cross-sectional view of a cerclage wire (“cable”) tensioning device 600, according to an exemplary embodiment. As shown, the device 600 includes an elongated body 605, which is coupled to a base 610 at a first end. The body 605 includes a channel or cannula 620, which is disposed within the body 605 and extends substantially along a length of the body 605. The body 605 includes an opening 615 at a second end, which is opposite the first end and distally positioned relative to the base 610. Adjacent to the channel 620, there is a track 617, which facilitates slidable displacement of a unidirectional locking mechanism 623 (e.g., ratchet mechanism, ratchet and pawl mechanism, spring swage mechanism, etc.) relative to the track 617. The mechanism 623 may be retained within the body 605 adjacent the track 617 via one or more grooves, clips, lips, ridges, or other equivalent features known in the art. As shown, the mechanism 623 includes a first edge 625 and a second edge 630, which are positioned adjacent opposing inner walls of the body 605. Between the edges, 625, 630, the mechanism 623 includes a locking member 635 (e.g., a wedge), which includes an engagement region configured to engage with a cable disposed within the channel 620. The locking member 635 also include a pin joint 640, which allows passage of cable in a first direction and prevents passage of cable in a second direction opposite the first direction.

Accordingly, during use of the device 600, cable may be inserted within the channel 620 of the body 605. Once the cable is engaged with the locking member 635, the locking mechanism 623 may be slidably displaced relative to the track 617 such that the locking mechanism 623 moves toward the base 610. As the locking mechanism 623 is moved toward the base 610, the locking member 635 engages with cable within the channel 620 and generates tension in the cable. Once the cable is tensioned, or to apply additional tension to the cable, the locking mechanism 623 may be slidably displaced away from the base 610. Accordingly, the locking member 635 may rotate about the pint joint 640 to enable the locking mechanism 623 to move relative to the cable within the body 605. In some embodiments, the device 600 may be structured such that displacement of the locking mechanism 623 within the body 605 is controlled by a spring mechanism 645, as shown in FIG. 15. The spring mechanism 645 may include one or more springs 647, which are coupled between the locking mechanism 623 and a ratcheting assembly 667. The ratcheting assembly 667 includes a wedge or other angled protruding member 675, which is configured to engage with a toothed track 670 such that the member 675 prevents displacement of the track 670 in a first direction and allows displacement of the track 670 in a second direction opposite the first direction. Displacement of the member 675 relative to the track 670 causes displacement of the locking mechanism 623 as facilitated by the spring mechanism 645. In various embodiments, the ratcheting assembly 667 includes a ratchet wheel to facilitate advancement of the member 675 relative to the track 670.

As shown in FIG. 15, the device 600 may include an actuation mechanism 650, which is coupled to the body 605. The actuation mechanism 650 includes a handle 655, which includes a threaded member 660 disposed therein, where the threaded member 660 is configured to engage with the track 670 of the ratcheting assembly 667 at an interface 665. Accordingly, during use of the device 600, a cable may be received (e.g., from a cable passing device such as device 100, 200, 300, 400, or 500) and inserted within the channel 620 of the body 605. The handle 655 of the actuation mechanism 650 may then be rotated to cause engagement of the threaded member 660 and the track 670, which may cause advancement of the track 670 relative to the member 675. Advancement of the track 670 results in tensioning of the cable by the locking mechanism 623 via the spring mechanism 645, where the member 675 prevents loosening of the cable from within the device 600.

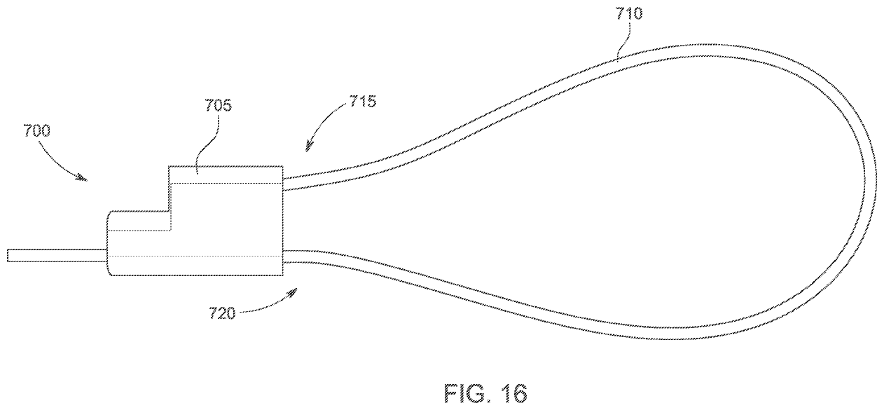

Once cable from a cable passing device (e.g., device 100, 200, 300, 400, and/or 500) is tensioned by a cable tensioning device (e.g., device 600), the cable may be secured during a cable securement device. FIG. 16 shows a cable securement device 700, according to an exemplary embodiment. The cable securement device 700 includes a body 705, which includes one or more cinching mechanisms therein, which may be operated to secure a cable 710 after the cable has been passed through a surgical site and tensioned. As shown, the body 705 may fixedly secure the cable 710 at a first end 715 and a second end 720, where tension within the cable 710 may be maintained or released using one or more set screws disposed within the body 705. In other embodiments, the cable 710 may be retained within the body 705 by crimping the body 705 and/or by using one or more pins.

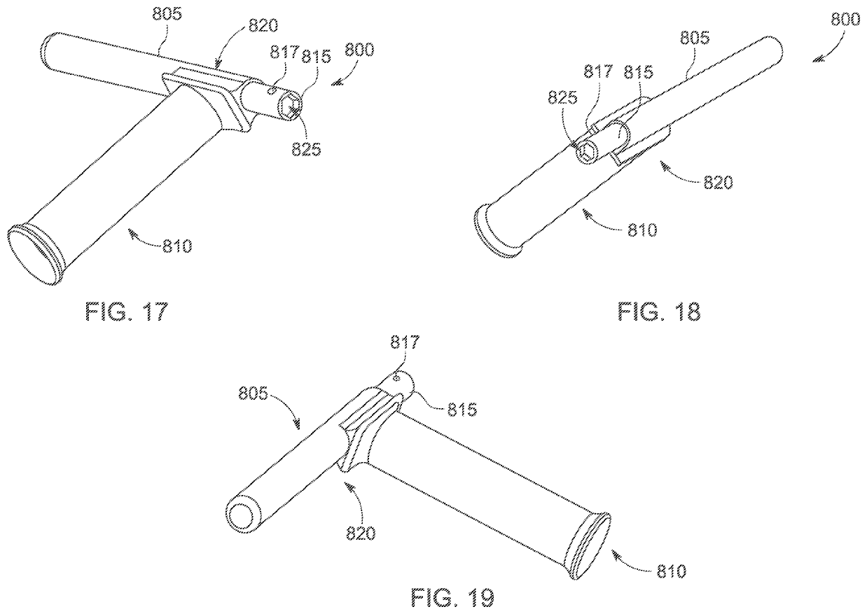

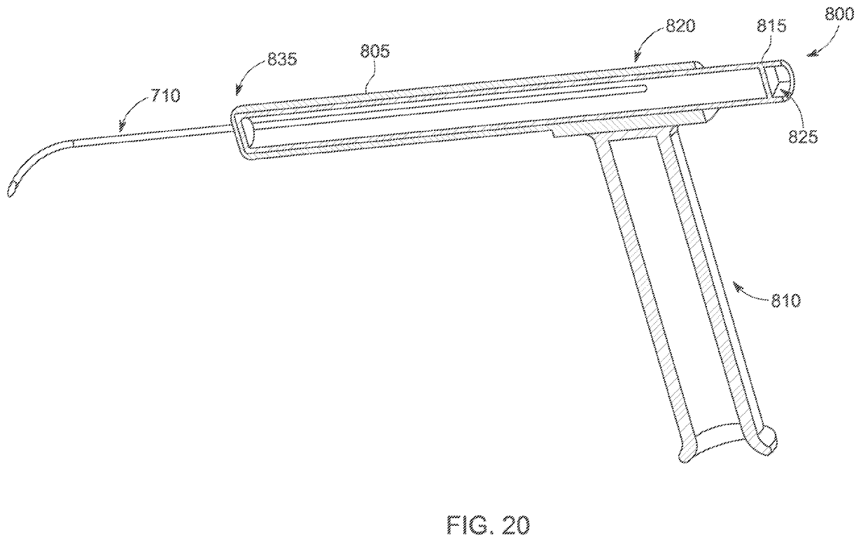

Once cable has been passed (e.g., using device 100, 200, 300, 400, and/or 500), tensioned (e.g., using device 600), and secured (e.g., using device 700), cable can be cut using a cable cutting device. FIGS. 17-19 show alternate perspective views of a cerclage wire (“cable”) cutting device 800, according to an exemplary embodiment. As shown, the device 800 includes a first shaft 805 and a coaxial second shaft 815, which is configured to rotate relative to the first shaft 805. The second shaft 815 includes a hole or aperture 817 through an outer wall of the shaft 815. In various embodiments, the hole 817 has a diameter of approximately 8 mm. The device 800 also includes a handle 810, which is coupled to or integrally formed with the first shaft 805. The first shaft 805 and the second shaft 815 define an inner channel 825. Accordingly, cable may be provided through the hole 817 of the second shaft 815 and slack within the cable may extend through the channel 825. The handle 810 may then be secured (e.g., held by a user) to prevent movement of the first shaft 805. With the cable extending through the hole 817 of the second shaft 815, the second shaft 815 may be rotated relative to the first shaft 805 to then cut cable extending through the device 800. In various embodiments, rotation of the second shaft 815 may be manually or automatically driven (e.g., by a motor). In embodiments, rotation of the second shaft 815 is driven by one or more motors, which may be controlled by one or more controllers. The shaft 805 may include a cutting interface disposed within a distal end 835, as shown in FIG. 20. Accordingly, the cable 710 may be cut at the end 835 after it has been inserted through the aperture 817 and the shaft 815. In various embodiments, the cutting interface within the distal end 835 may include one or more blades that may rotate, slide, or otherwise articulate to facilitate cutting of the cable 710 within the device 800.

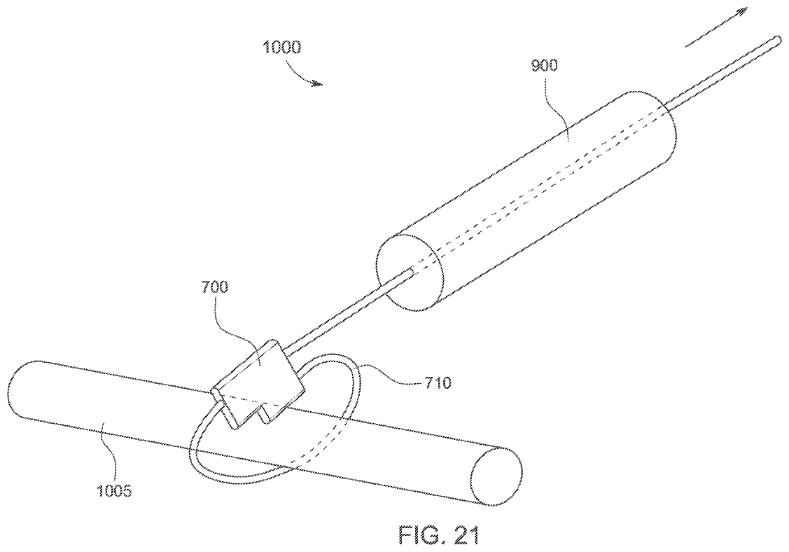

In various implementations, medical personnel (e.g., orthopedic surgeon or an associate thereof) may acquire a cable passing device (e.g., device 100, 200, 300, 400, 500) and insert cerclage wire (“cable”) into the cable passing device. In various embodiments, the cable passing device may be provided in multiple sizes, where a size of the cable passing device used in surgical procedures is chosen based on a particular application. For example, the cable passing device may be provided in small, medium, or large sizes. In another example, the cable passing device may be sized based on pediatric, geriatric, or other applications. The cable passing device may then be inserted within a surgical site and maneuvered such that the cable passing device may circumnavigate one or more anatomical features within the surgical site. The cable may then be advanced through the cable passing device and thus through the surgical site. The cable passing device may then be extracted from the surgical site. In various embodiments, the cable insertion point and the cable exit point are disposed within the cable passing device such that the cable is inserted and removed from the cable passing device at locations disposed outside the body to facilitate minimal invasiveness of the surgical procedure. A cable tensioning device (e.g., device 600), which may be coupled to the cable passing device or provided separately, may then receive the cable from the surgical site, where the cable tensioning device may then cause tension within the cable. A cable securement device (e.g., device 700), which may be coupled to at least one of the cable passing device or the cable tensioning device or may be provided separately, may then receive the cable and secure terminal ends of the cable therein. A free end of the secured cable may then be received within a cable cutting device (e.g., device 800), which may be coupled to one or more of the aforementioned devices or may be provided separately, may then cut the cable such that the secured cable may fix or stabilize the one or more anatomical features within the surgical site. For example, as in FIG. 21, which shows a cerclage assembly 1000, the cable securement device 700 may be used in conjunction with a device 900, where the device 900 is configured to both tension the cable 710 and cut the cable 710. Accordingly, the device 700 may first be used to secure the cable 710 about an anatomical feature 1005 (e.g., femur, humerus, etc.) within a surgical site, and the device 900 may be used to receive a terminal end of the cable 710 to first tension and then subsequently cut the cable 710 to stabilize the anatomical feature 1005.

Notwithstanding the embodiments described above in reference to FIGS. 1-21, various modifications and inclusions to those embodiments are contemplated and considered within the scope of the present disclosure.

As utilized herein with respect to numerical ranges, the terms “approximately,” “about,” “substantially,” and similar terms generally mean+/−10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A device for passing cerclage wire, the device comprising:
a first member having a first region and a second region, the first member having a cannula formed therein, the first region being substantially linear in shape and the second region being hooked; and
a second member having a third region and a fourth region, the second member having a second cannula formed therein, the third region being substantially linear in shape and the fourth region being hooked,
wherein the first region includes at least one aperture that is fluidly connected to the cannula and configured to receive cerclage wire,
wherein the third region includes at least one aperture fluidly connected with the second cannula.

2. The device of claim 1, wherein a terminal end of the second region is configured to be received within a terminal end of the fourth region.

3. The device of claim 1, wherein a terminal end of the second region is configured to receive a terminal end of the fourth region.

4. The device of claim 1, further comprising a telescoping member coaxially disposed in an overlapping arrangement with one of the second region or the fourth region in an open configuration, the telescoping member being slidably displaceable from the one of the second region or the fourth region to at least partially overlap with the other of the one of the second region or the fourth region in a closed configuration.

5. The device of claim 4, wherein the telescoping member is configured to slidably displace through approximately 90 to 270 degrees.

6. The device of claim 4, wherein the telescoping member is configured to slidably displace through up to 360 degrees.

7. The device of claim 4, wherein the telescoping member is coupled to a linear actuator, the linear actuator being disposed along the first member and configured to slide relative to the first member, and wherein displacement of the linear actuator causes displacement of the telescoping member.

8. The device of claim 7, wherein the linear actuator extends between the first region and the second region.

9. The device of claim 1, further comprising first handle connected to the first member and a second handle connected to the second member, where user interaction with the first handle and the second handle linearly positions the first member and the second member, placing the device alternately in an open configuration and a closed configuration.

10. The device of claim 1, further comprising a locking mechanism disposed between the first region and the third region, the locking mechanism comprising:
a grooved member coupled to the first region; and
a protruding member coupled to the third region, and configured to be received within the grooved member, where the protruding member is slidably engaged with the grooved member.

11. The device of claim 1, further comprising a joint pivotably coupling the first member and the second member, wherein the first member connects to the pivot between the first region and the second region, and wherein the second member connects to the pivot between the third region and the fourth region.

12. The device of claim 11, further comprising at least one handle rotatably coupled to the joint, wherein rotation of the at least one handle causes the first member and the second member to pivot about the joint such that a distance between a distal end of the first member and a distal end of the second member changes.

13. A surgical kit for stabilizing one or more anatomical features within a surgical site, the kit comprising:
a first device for passing cerclage wire, the first device comprising:
a first cannulated member having a first region and a second region; and
a second cannulated member having a third region and a fourth region;
wherein each of the first and third regions are substantially linear in shape and each of the second and fourth regions are hooked, each of the first and third regions including at least one aperture disposed therein and configured to receive cerclage wire;
a second device for tensioning cerclage wire, the second device comprising:
a body having an inner channel disposed therein; and
a locking mechanism disposed within the channel configured to slidably displace within the channel along an axis of the body;
wherein displacement of the locking mechanism is configured to tension a cerclage wire; and
a third device for cutting cerclage wire, the third device comprising:
a first shaft;
a second shaft coaxially disposed within the first shaft; and
a handle coupled to the first shaft;
wherein a distal end of the first shaft includes a cutting interface configured to cut cerclage wire passing through the first shaft and the second shaft.

14. The surgical kit of claim 13, further comprising a fourth device for securing cerclage wire.

15. The surgical kit of claim 13, wherein the second device is disposed within the third device.

16. A method for stabilizing anatomy within a surgical site, the method comprising:
providing a first device, wherein the first device comprises:
an elongated first member having a substantially linear first region and a hooked second region, the elongated first member having a cannula formed therein in fluid communication with at least one aperture, wherein the at least one aperture is configured to receive cerclage wire; and
an elongated second member having a substantially linear third region and a hooked fourth region, the elongated second member having a second cannula formed therein
passing a cerclage wire through the surgical site using the first device by carrying out operations comprising:
inserting the elongated member of the first device into the surgical site;
circumnavigating the second region of the first member about an anatomical feature within the surgical site;
inserting the cerclage wire through the at least one aperture within the first region of the elongated member of the first device;
advancing the cerclage wire through the cannula of the first device; and removing the first device from the surgical site;
providing a second device, wherein the second device comprises:
a body having an inner channel disposed therein; and
a locking mechanism disposed within the channel configured to slidably displace within the channel along an axis of the body;
passing a terminal end of the cerclage wire into the locking mechanism of the second device;
creating a tension within the cerclage wire by displacing the locking mechanism within the second device, wherein the locking mechanism includes a ratchet and a pawl configured to induce the tension within the cerclage wire;
securing a first portion adjacent the terminal end of the cerclage wire using a third device; and
cutting the terminal end of the cerclage wire using a fourth device, the fourth device comprising:
a first shaft;
a second shaft coaxially disposed within the first shaft; and
a handle coupled to the first shaft;
wherein a distal end of the first shaft includes a cutting interface configured to cut the cerclage wire passing through the first shaft and the second shaft.
